(19) United States
(12) Patent Application Publication
(10) Pub. No.: US 2024/0389894 A1
(43) Pub. Date: Nov. 28, 2024

(54) ANALYTE DETECTION DEVICE COMMUNICATION SYSTEM AND METHOD FOR REALIZING COMMUNICATION CONNECTION OF ANALYTE DETECTION DEVICE

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 18/693,956

(22) PCT Filed: Jun. 17, 2022
(86) PCT No.: PCT/CN2022/099387

§ 371 Date: Mar. 21, 2024

(30) Foreign Application Priority Data
Sep. 27, 2021 (WO) PCT/CN2021/120856

(51) Int. Cl. A61B 5/145; A61B 5/00

(52) U.S. Cl. CPC A61B 5/14532; A61B 5/14503; A61B 5/7405; A61B 5/742; A61B 5/7455; A61B 2560/045; A61B 2560/0462

(57) ABSTRACT

System and method for realizing communication connection of analyte detection device are disclosed. When the communication connection is not established and the communication connection has been established, the analyte detection device transmits two different signals respectively, namely the first signal and the second signal. The remote equipment searches and identifies the nearby signals, and executes different communication connection procedures according to the signals. When only one first signal is recognized by the remote equipment, it can be judged that the analyte detection device transmitting the signal is the analyte detection device to be established with communication connection, and the communication connection can be established directly without the user manually inputting or scanning the equipment code, which simplifies the process of establishing the communication connection, avoids the user inputting or scanning the wrong equipment code, and improves the user experience.

### TECHNICAL FIELD

The invention mainly relates to the field of medical devices, in particular to a system and method for realizing the communication connection of analyte detection device.

### BACKGROUND

The pancreas in a normal human body can automatically monitor the blood glucose level and automatically secrete required amount of insulin/glucagon. In the body of a type 1 diabetes patient, the pancreas does not function properly and cannot produce enough insulin for the body. Therefore, type I diabetes is a metabolic disease caused by abnormal pancreatic function, and diabetes is a lifelong disease. At present, there is no cure for diabetes with medical technology. The onset and development of diabetes and its complications can only be controlled by stabilizing blood glucose.

Diabetics need to have their blood glucose measured before they inject insulin into the body. At present, most of the testing methods can continuously measure blood glucose level and transmit the data to a remote device in real time for the user to view. This method is called Continuous Glucose Monitoring (CGM).

When the analyte detection device of the prior art establishes the communication connection with the remote equipment, the user needs to manually input or scan the equipment code of the analyte detection device on the remote equipment. The process is cumbersome. When the equipment code is blurred or falls off, the input or scanning of the equipment code is easy to make mistakes and affect the user's use experience.

Therefore, the prior art urgently needs a communication system and method of analyte detection device that can establish communication connection without equipment code.

### BRIEF SUMMARY OF THE INVENTION

The invention discloses a system and method for realizing the communication connection of analyte detection device, comprising the analyte detection device and the remote equipment. The analyte detection device without communication connection transmits the first signal, the analyte detection device with communication connection transmits the second signal, and the remote equipment is used to search and recognize nearby signals, and execute different communication connection procedures according to the signals, which simplify the process of establishing communication connection and improve the user experience.

The embodiment of the invention discloses an analyte detection device communication system, which comprises: the analyte detection device, which is installed on the surface of the user's skin, obtains the analyte parameter information in the user's body, and transmits signals to the remote equipment, wherein the signals at least comprise the first signal transmitted by the analyte detection device without establishing communication connection and the second signal transmitted by the analyte detection device with established communication connection. And the remote equipment, which is used to search and recognize nearby signals and execute communication connection procedures.

According to one aspect of the invention, the remote equipment is configured to execute the first communication connection program when the remote equipment recognizes only one first signal: the remote equipment establishes a communication connection with the analyte detection device transmitting the first signal.

According to one aspect of the invention, before executing the first communication connection program, the remote equipment sends a prompt that needs to be confirmed by the user.

According to one aspect of the invention, the remote equipment is configured to execute the second communication connection program when the remote equipment recognizes at least two first signals: prompt the user to input the equipment code of the analyte detection device to be connected.

According to one aspect of the invention, the remote equipment is configured to execute the third communication connection program when the remote equipment recognizes at least two first signals: prompt the user to change the operation place until the remote equipment recognizes only one first signal, and then execute the first communication connection program.

According to one aspect of the invention, the effective range of the first signal is 0˜10 m, and the effective range of the second signal is 0˜10 m.

According to one aspect of the invention, the effective range of the first signal is 0˜1 m, and the effective range of the second signal is 0˜10 m.

According to one aspect of the invention, the remote equipment is configured to send an alarm or fault prompt to the user when the remote equipment does not recognize the first signal.

According to one aspect of the invention, the prompt is in the form of one or more of audio, video or vibration.

According to one aspect of the invention, the first signal differs from the second signal in signal frequency, signal type, signal strength or/and signal format.

The embodiment of the invention also discloses a method for realizing the communication connection of analyte detection device, comprising providing analyte detection device, the analyte detection device without communication connection transmits the first signal, and the analyte detection device with communication connection transmits the second signal. And remote equipment, which is used for communication connection with analyte detection device. Activate the analyte detection device to establish communication connection, transmit the first signal, start the remote equipment, search and recognize the nearby signals, and execute the communication connection program.

According to one aspect of the invention, the remote equipment is configured to execute the first communication connection program when the remote equipment recognizes only one first signal: the remote equipment establishes a communication connection with the analyte detection device transmitting the first signal.

According to one aspect of the invention, before executing the first communication connection program, the remote equipment sends a prompt that needs to be confirmed by the user.

According to one aspect of the invention, the remote equipment is configured to execute the second communication connection program when the remote equipment recognizes at least two first signals: prompt the user to input the equipment code of the analyte detection device to be connected.

According to one aspect of the invention, the remote equipment is configured to execute the third communication connection program when the remote equipment recognizes at least two first signals: prompt the user to change the operation location until the remote equipment recognizes only one first signal, and then execute the first communication connection program.

According to one aspect of the invention, the remote equipment is configured to send an alarm or fault prompt to the user when the remote equipment does not recognize the first signal.

According to one aspect of the invention, the first signal differs from the second signal in signal frequency, signal type, signal strength or/and signal format.

Compared with the prior art, the technical scheme of the invention has the following advantages:

In the system and method for realizing the communication connection of the analyte detection device disclosed by the invention, the analyte detection device transmits two different signals, namely the first signal and the second signal, when the communication connection is not established and the communication connection is established. When the remote equipment only recognizes the signal transmitted by the analyte detection device without the communication connection, it can be judged that the analyte detection device transmitting the signal is the analyte detection device to be established with communication connection, and the communication connection can be established directly without the user manually inputting or scanning the equipment code, which simplifies the process of establishing the communication connection, avoids the user inputting or scanning the wrong equipment code, and improves the user experience.

Further, in a complex environment, if the remote equipment recognizes the signals transmitted by more than one analyte detection device that has not established a communication connection, such as multiple first signals, or multiple first signals and second signals, the user is prompted to carry the analyte detection device and the remote equipment to establish a communication connection to change the operation location until the remote equipment recognizes only one first signal, Judging that the signal is the signal sent by the analyte detection device to establish the communication connection, the communication connection can be established directly without the user manually inputting or scanning the equipment code, which simplifies the process of establishing the communication connection, avoids the user inputting or scanning the wrong equipment code, and improves the user experience.

Further, if the remote equipment does not recognize the first signal, it is judged that the analyte detection device to be established communication connection does not normally transmit the signal, and an alarm or fault prompt is sent to the user, which improves the user experience.

### BRIEF DESCRIPTION OF THE DRAWINGS

FIG. 1 is a schematic diagram of the structure of the analyte detection system according to an embodiment of the invention;

FIG. 2 is a schematic diagram of the analyte detection device according to an embodiment of the invention;

FIG. 3a is a structural schematic diagram of the wake-up module of the analyte detection device comprising a sensor according to an embodiment of the invention;

FIG. 3b is a schematic diagram of the wake-up module of the analyte detection device comprising a light sensing element according to an embodiment of the invention;

FIG. 4a is a structural schematic diagram of the analyte detection system comprising magnetic component and magnetic induction element according to an embodiment of the invention;

FIG. 4b is a structural schematic diagram of the wake-up module of an analyte detection device comprising a magnetic induction element according to an embodiment of the invention;

FIG. 4c is a schematic diagram of the wake-up module of the analyte detection device comprising a magnetic induction element according to an embodiment of the invention;

FIG. 5a is a structural schematic diagram of the analyte detection system comprising an acceleration sensor according to an embodiment of the invention;

FIG. 5b is a structural schematic diagram of the wake-up module of the analyte detection device comprising an acceleration sensor according to an embodiment of the invention;

FIG. 5c is a functional schematic diagram of the wake-up module of the analyte detection device comprising the acceleration sensor to an embodiment of the invention;

FIG. 6 is a schematic diagram of the communication connection between the analyte detection device and remote equipment according to the embodiment of the invention;

FIG. 7 is a schematic diagram of the process of establishing communication connection between the analyte detection device and an external device according to the embodiment of the invention.

### DETAILED DESCRIPTION

As mentioned above, when the analyte detection device of the prior art establishes a communication connection with the remote equipment, the user needs to manually input or scan the equipment code of the analyte detection device on the remote equipment. The process is cumbersome. When the equipment code is blurred or falls off, the input or scanning of the equipment code is prone to error, which affects the user's use experience.

In order to solve this problem, the invention provides a system and method for realizing the communication connection of the analyte detection device. When the communication connection is not established and the communication connection has been established, the analyte detection device transmits two different signals, namely the first signal and the second signal respectively. When the remote equipment only recognizes the first signal, It can be judged that the analyte detection device transmitting the signal is the analyte detection device to be established with communication connection, and the communication connection can be established directly without the user manually inputting or scanning the equipment code, which simplifies the process of establishing the communication connection, avoids the user inputting or scanning the wrong equipment code, and improves the user experience.

Various exemplary embodiments of the invention will now be described in detail with reference to the attached drawings. It is understood that, unless otherwise specified, the relative arrangement of parts and steps, numerical expressions and values described in these embodiments shall not be construed as limitations on the scope of the present invention.

In addition, it should be understood that the dimensions of the various components shown in the attached drawings are not necessarily drawn to actual proportions for ease of description, e.g. the thickness, width, length or distance of some elements may be enlarged relative to other structures.

The following descriptions of exemplary embodiments are illustrative only and do not in any sense limit the invention, its application or use. Techniques, methods and devices known to ordinary technicians in the relevant field may not be discussed in detail here, but to the extent applicable, they shall be considered as part of this manual.

It should be noted that similar labels and letters indicate similar items in the appending drawings below, so that once an item is defined or described in one of the appending drawings, there is no need to discuss it further in the subsequent appending drawings.

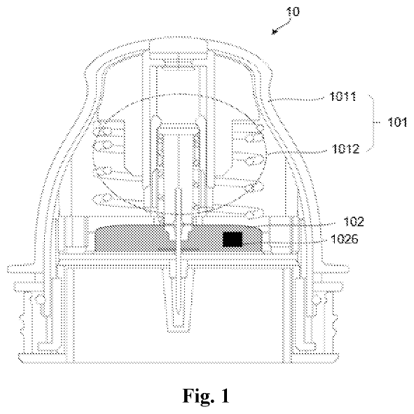

FIG. 1 is a structural diagram of the analyte detection system in an embodiment of the invention. The analyte detection system 10 comprises an auxiliary installer 101 and an analyte detection device 102. The auxiliary installer 101 comprises a housing 1011 and an auxiliary mounting module 1012, which is located inside the housing 1011. Analyte detection device 102 is located at the ejector end of auxiliary mounting module 1012, which enables rapid installation of analyte detection device 102 to the host skin surface when in use.

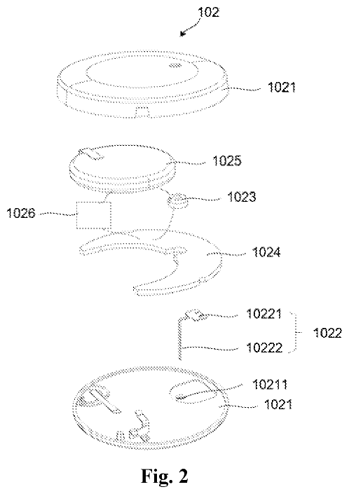

FIG. 2 is a schematic diagram of the analyte detection device to an embodiment of the invention. The analyte detection device 102 comprises the shell 1021, the sensor 1022, the transmitter 1023, the internal circuit 1024, the battery 1025 and the wake-up module 1026. Sensor 1022 comprises an external part 10221 and an internal part 10222. The external part 10221, transmitter 1023, internal circuit 1024, battery 1025 and wake-up module 1026 are located inside the shell 1021. The internal part 10222 passes through the through-via 10211 on the shell 1021 to the outside to puncture the host subcutaneous and detect the parameter information of analyte. What technicians in this field can know is that in order to pierce the internal part 10222 subcutaneous to the host, the through-via 10211 is located on the side of shell 1021 which is away from housing 1011, and at the same time, a tape (not shown in the figure) is arranged on the surface, which is used to attach the analyte detection device 102 to the skin surface of the host. The external part 10221 is electrically connected with the transmitter 1023 through the internal circuit 1024, which can transmit analyte parameter information to the outside equipment.

Before use, the shell 1021 of the analyte detection device 102 is releasable connected with the housing 1011 of auxiliary mounting device 101. Here, “releasable connection” means that shell 1021 is connected with housing 1011 by means of buckle, clamp, etc. Under the action of ejector mechanism of auxiliary mounting module 1012, the shell 1021 can be separated from housing 1011.

After the life of the sensor 1022 has expired, or the battery 1025 has run out of power, or other factors have caused the analyte detection device to fail, the user removes the entire analyte detection device from the skin surface of the host, discards it and replaces it with a new analyte detection device, is beneficial to maintain the best use of the parts of the device.

When analyte detection device 102 is installed on the skin surface of the host and starts to use, communication needs to be established with outside equipment such as PDM (Personal Diabetes Manager), mobile phone, etc., for data interaction, so as to transmit the detected analyte information data in the host to outside equipment.

As mentioned above, the analyte detection device 102 is in dormant state and transmits signal to the outside equipment at the first frequency until communication is formally established with the outside equipment. In the embodiment of the invention, the analyte detection device 102 transmits signal at a lower first frequency to an outside equipment in dormant state to reduce battery energy consumption. In the more preferred embodiment of the invention, the first frequency is 0˜12 times/hour. In the more preferred embodiment of the invention, the first frequency is 0 times/hour, that is, the analyte detection device 102 does not transmit signal to the outside equipment in dormant state.

In order to establish communication between the analyte detection device 102 which is in dormant state and outside equipment, wake-up module 1026 wakes up analyte detection device 102 according to triggering conditions, so that it enters the working state and transmits signal to the outside equipment with the second frequency, and then communication is established after the outside equipment responds. The second frequency is higher than the first frequency in order to obtain analyte parameter information conveniently and in real time. In the preferred embodiment of the invention, the second frequency is 12˜3600 times/hour. In a more preferred embodiment of the invention, the second frequency is 30 times/hour.

### First Embodiment

### Light Sensing Element

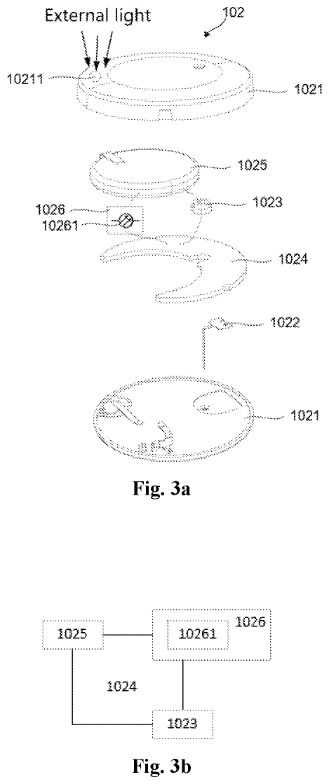

FIG. 3a is a schematic diagram of the structure of the wake-up module of the analyte detection device comprising a light sensing element in an embodiment of the invention. FIG. 3b is a functional schematic diagram of the wake-up module of the analyte detection device comprising the light sensing element in an embodiment of the invention.

In the embodiment of the invention, the wake-up module 1026 comprises a light sensing element 10261, such as photoelectric switch, which is in open state when there is no light beam or weak light beam irradiation and in a closed state when there is light beam irradiation.

In combination with FIG. 1 and FIG. 3b, transmitter 1023 is connected with battery 1025 through internal circuit 1024, forming a closed loop. The circuit is connected with a wake-up module 1026, which is connected with a light sensing element 10261 inside. The triggering condition of the wake-up module 1026 is the light intensity change received by the light sensing element 10261. In the preferred embodiment of the invention, the triggering condition of the wake-up module 1026 is that the light intensity received by the light sensing element 10261 changes from weak to strong.

In the embodiment of the invention, the analyte detection device 102 is not separated from the auxiliary mounting device 101 before it is installed on the skin surface of the host, and the shell 1021 and housing 1011 form a closed and opaque space. Since the light-transmitting area 10211 is located near the end of the housing 1011, there is no external light irradiates on light sensing element 10261, battery 1025 supplies power to transmitter 1023 through wake-up module 1026 (comprising light sensing element 10261), light sensing element 10261 is in open state, and thus the transmitter 1023 is in dormant state, and analyte detection device 102 transmits signal to outside equipment at the first frequency. After the analyte detection device 102 is installed on the skin surface of the host through the auxiliary mounting module 1012, the shell 1021 is separated from the housing 1011, and the external light can be irradiated to the light sensing element 10261 through the shell 1021. The light sensing element 10261 is in closed state. The transmitter 1023 enters the working state, and the analyte detection device 102 transmits signal to the outside equipment at the second frequency. After the response of the outside equipment, the communication is established and the analyte detection data is transmitted to the outside equipment.

In the embodiment of the invention, the shell 1021 is made of light transmittance material, such as one of polymethyl methacrylate (PMMA), polystyrene (PS), polycarbonate (PC) or poly 4-methyl-1-pentene (TPX), and the light transmittance of these material is 40%˜95%. After the separation of shell 1021 and housing 1011, the external light can be irradiated on the light sensing element 10261 through the shell 1021.

In other embodiment of the invention, the shell 1021 comprises light-transmitting area 10211, the light transmittance of the light-transmitting area 10211 is higher than that of the shell 1021, so that more external light is irradiated on the light sensing element 10261, the light intensity variation of the light sensing element 10261 is increased, and the reliability of the light sensing element 10261 is improved.

In another embodiment of the invention, the light-transmitting area 10211 comprises at least one light-transmitting hole, or an array combination of several light-transmitting holes. The light-transmitting hole can make more external light illuminate on the light sensing element 10261, further increase the light intensity variation of the light sensing element 10261, and improve the reliability of the light sensing element 10261. A light-transmittance film is arranged in the light-transmitting hole (not shown in the figure out), which can prevent external water droplets, dust and other dirt from inputting the analyte detection device through the light-transmitting hole and improve the reliability of the device.

In the embodiment of the invention, the light sensing element 10261 can sense visible light or invisible light, such as infrared or ultraviolet light. In the preferred embodiment of the invention, the light sensing element 10261 senses visible light so that the user can wake up the analyte detection device indoors or outdoors.

In other embodiment of the invention, the switch condition of open circuit and closed circuit of the light sensing element is low light irradiation to strong light irradiation, that is, before the separation of shell 1021 and housing 1011, weak external light is allowed to illuminate the interior of housing 1011, and the light sensing element 10261 receives weak light, but it is still in open circuit and the transmitter 1023 is in dormant state, which takes into account that the actual connection between shell 1021 and housing 1011 is not completely sealed. When the shell 1021 is separated from the housing 1011, the external light completely irradiates on the light sensing element 10261 through the shell 1021, and the light intensity received by the light sensing element 10261 becomes stronger. After reaching the set light intensity threshold, the light sensing element 10261 switches to the closed state, and the transmitter 1023 enters the working state to transmit signal to the outside equipment at the second frequency. After the response from the outside equipment, the communication is established and the analyte detection data is transmitted to the outside equipment.

### Second Embodiment

### Magnetic Component and Magnetic Induction Element

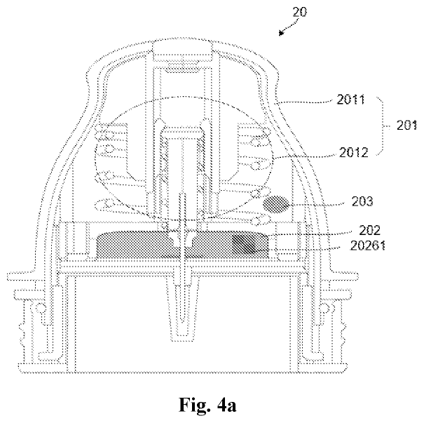

FIG. 4a is a schematic diagram of the structure of the analyte detection system comprising magnetic component and magnetic induction element in an embodiment of the invention. FIG. 4b is a schematic diagram of the structure of the wake-up module of the analyte detection device comprising the magnetic induction element in an embodiment of the invention. FIG. 4c is a schematic diagram of the function of the wake-up module of the analyte detection device comprising the magnetic induction element in an embodiment of the invention.

In the embodiment of the invention, a magnetic component 203 is arranged on the housing 2011, and a magnetic induction element 20261 is arranged in the wake-up module 2026, the battery 2025 supplies power to transmitter 2023 through the wake-up module 2026 (comprising the magnetic induction element 20261). Magnetic component 203 provides a stable magnetic field, and magnetic induction element 20261 is located in the magnetic field of magnetic component 203 and induces the magnetic field of magnetic component 203 to generate a signal. The triggering condition of the wake-up module 2026 is the magnetic field change induced by the magnetic induction element 20261.

The transmitter 2023 is connected with the battery 2025 through the internal circuit 2024, forming a closed loop, and the circuit is connected with the wake-up module 2026. Before the analyte detection device 202 is installed on the skin surface of the host, the analyte detection device 202 is not separated from the auxiliary mounting device 201, and the relative position is fixed. The magnetic field induced by the magnetic induction element 20261 to the magnetic component 203 is stable. Under the stable magnetic field, the magnetic induction element 20261 is in the open state, the transmitter 2023 is in dormant state, and analyte detection device 202 transmits signal to outside equipment at the first frequency. After the analyte detection device 202 is installed on the skin surface of the host through the auxiliary mounting module 2012, the shell 2021 is separated from the housing 2011, and the distance between the magnetic induction element 20261 and the magnetic component 203 changes, so the induced magnetic field also changes, and the magnetic induction element 20261 switches to the closed state, and transmitter 2023 enters the working state. Analyte detection device 202 transmits signal to the outside equipment at the second frequency, and then establishes communication with outside equipment after the response of the outside equipment, and transmits analyte detection data to the outside equipment.

In the embodiment of the invention, the magnetic induction element 20261 senses the magnetic field strength or magnetic field direction of the magnetic component 203. Preferably, the induction element 20261 comprises a hall element (not shown in the figure out) that sensitively sensitizes the magnetic field strength of the magnetic component 203.

In the embodiment of the invention, the magnetic component 203 may be an individual part independent of the housing 2011, or a part of the housing 2011 which is embedded in the housing 2011.

In other embodiments of the invention, the housing 2011 is embedded or enclosed with a magnetic field shielding device (not shown in the figure out), such as a Faraday cage. Technicians in this field can know that the magnetic shielding device is located outside the magnetic component 203 to reduce the impact of external magnetic field on the magnetic induction element 20261.

### Third Embodiment

### Acceleration Sensor

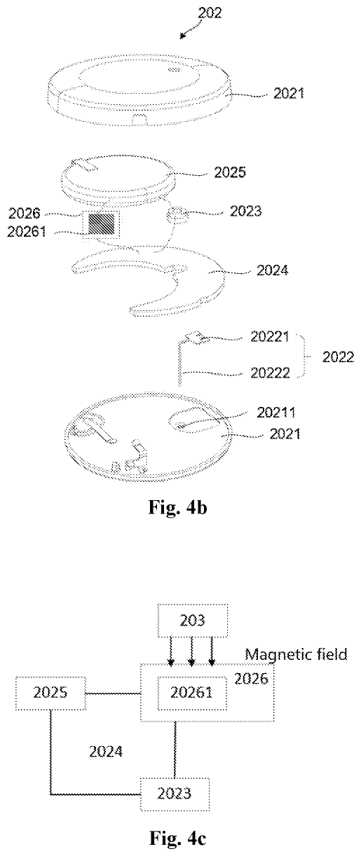

FIG. 5a is a schematic diagram of the structure of the wake-up module of the analyte detection system comprising the acceleration sensor in an embodiment of the invention. FIG. 5b is a schematic diagram of the structure of the wake-up module of the analyte detection device comprising the acceleration sensor in an embodiment of the invention. FIG. 5c is a schematic diagram of the function of the wake-up module of the analyte detection device comprising the acceleration sensor in an embodiment of the invention.

In the embodiment of the invention, the wake-up module 3026 comprises an acceleration sensor 30261, which can sensitively sense the values of motion parameters such as acceleration and adjust the circuit state of the wake-up module 3026 accordingly. The triggering condition of wake-up module 3026 is the motion parameter change of acceleration sensor 30261.

Transmitter 3023 is connected with battery 3025 through internal circuit 3024 to form a closed loop, and the circuit is connected with the wake-up module 3026, the battery 3025 supplies power to transmitter 3023 through wake-up module 3026 (comprising acceleration sensor 30261). Before the analyte detection device 302 is installed on the skin surface of the host, the analyte detection device 302 and the auxiliary mounting device 301 are relatively fixed. In order to pierce the internal part of the sensor 30222 of the analyte detection device into the skin of the host and reduce the pain sensation during the stabbing, the auxiliary mounting module 3012 adopts ejector mechanism 30121. Such as spring and other elastic parts, through the auxiliary needle 30122 can quickly pierce the body part 30222 into the host subcutaneous. When the ejector mechanism 30121 is in use, it produces a large instantaneous forward acceleration a1, and when it is installed on the skin surface of the host, it is obstructed by the skin to produce a reverse acceleration a2. After the acceleration sensor 30261 senses the above two accelerations, it can be determined that the analyte detection device 302 is installed on the skin surface of the host.

In the embodiment of the invention, before the analyte detection device 302 is installed on the skin surface of the host, the wake-up module 3026 is in an open state, and the transmitter 3023 is in a dormant state and transmits signal to the outside equipment at the first frequency. Acceleration sensor 30261 determines that the analyte detection device 302 is installed on the skin surface of the host, and the wake-up module 3026 switches to the closed state, and transmitter 3023 enters the working state and transmits signal to the outside equipment at the second frequency. After the response of the outside equipment, the communication is established and the analyte detection data is transmitted to the outside equipment.

It can be understood by those skilled in the art that “first frequency” and “second frequency” in this patent refer to the transmission frequency of the signal to characterize the transmission interval of the signal.

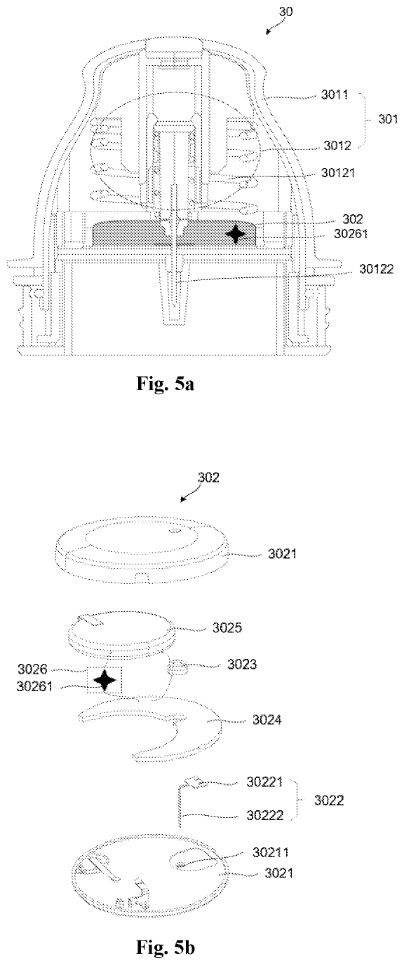

FIG. 6 is a schematic diagram of the communication connection between the analyte detection device and remote equipment according to the embodiment of the present invention.

In the embodiment of the invention, the analyte detection device 102 is activated after inputting the working state, transmits the first signal before establishing communication with the remote equipment 103, and transmits the second signal after establishing communication with the remote equipment 103.

In some embodiments of the invention, the first signal uses low-power Bluetooth and the second signal uses near-field communication (NFC). Or the first signal uses WiFi and the second signal uses low-power Bluetooth.

In other embodiments of the invention, the first signal and the second signal are of the same type, but their signal strengths are different. For example, the signal strength of the first signal is weaker than that of the second signal. In the preferred embodiment of the invention, the strengths of the first signal and the second signal are set so that the effective range of the first signal is 0˜10 m and the effective range of the second signal is 0˜10 m. In the more preferred embodiment of the invention, the effective range of the first signal is 0˜1 m, and the smaller effective range of the first signal is convenient for the external device 103 to filter the fault first signal.

In other embodiments of the invention, the first signal and the second signal have different signal formats, for example, the communication connection status of the first signal packet is marked as A, and the communication connection status of the second signal packet is marked as B. The above mark can be located at any position of the packet, such as the packet header or the packet body, and so on. In a preferred embodiment of the invention, the packet header is set as the communication connection status flag bit. When the communication connection is not established, the first signal packet sent by the analyte detection device is A ••• while the communication connection is established, the second signal packet sent by the analyte detection device is B •••. In another preferred embodiment of the invention, a plurality of flag bits are set as communication connection status mark. In the state of no communication connection, the first signal packet sent by the analyte detection device is A ••• A ••• A •••, and in the state of established communication connection, the second signal packet sent by the analyte detection device is B ••• B ••• B •••. As long as the communication connection status of the analyte detection device can be distinguished, the number of flag bits and format of the data packet are not limited. In this embodiment, the form of the communication connection status mark A (B) in the data packet can be a single byte, such as 0 (1), or a multi byte, such as 000 (111), which is not limited here.

In other embodiments of the invention, the first signal and the second signal have different signal frequencies, for example, the first signal is the low-frequency signal and the second signal is the high-frequency signal, or the first signal is the high-frequency signal and the second signal is the low-frequency signal. Wherein, the first signal is the low-frequency signal and the second signal is the high-frequency signal, which is only used to explain that the frequency of the first signal is lower than the second signal, rather than to limit the specific frequency of the first signal and the second signal. Similarly, the first signal is the high-frequency signal and the second signal is the low-frequency signal, which is only used to explain that the frequency of the first signal is higher than the second signal, rather than to limit the specific frequency of the first signal and the second signal.

In other embodiments of the invention, the difference between the first signal and the second signal lies in the signal format and signal strength, or in the signal format and signal strength, the signal frequency, or a combination of other signal difference forms. The combination of various different forms of signals is more conducive to the remote equipment to distinguish the analyte detection device to be connected.

As long as the first signal can be distinguished from the second signal, the technical scheme comprising but not limited to the above can be adopted, which is not limited here. No matter how the first signal and the second signal are distinguished, the characteristics of the first signal and the second signal are pre stored in the remote equipment 103.

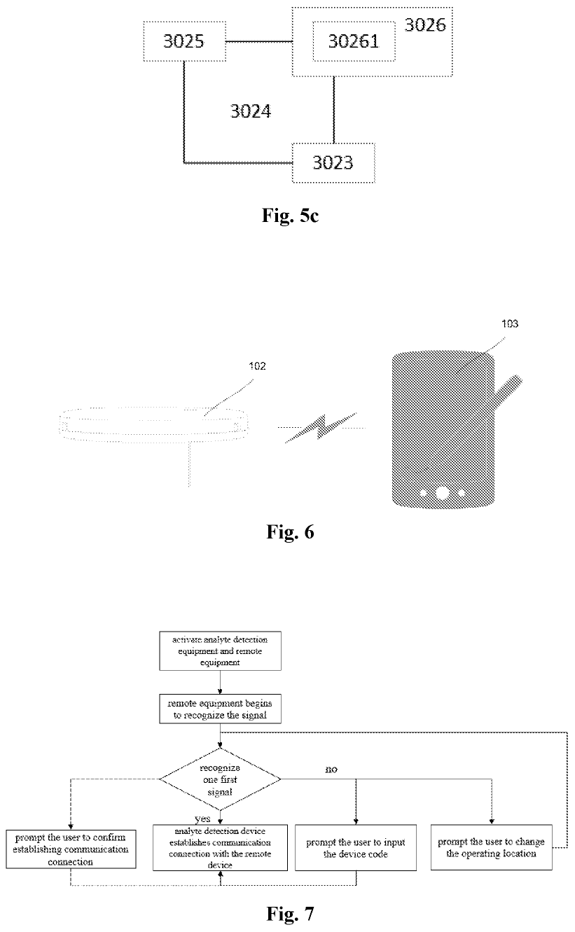

FIG. 7 is a schematic diagram of the process of establishing communication connection between the analyte detection device and an external device in an embodiment of the invention. Refer to the FIG. 7, in the embodiment of the invention, when the user needs to establish a communication connection between the analyte detection device 102 and the remote equipment 103, first place the remote equipment 103 near the analyte detection device 102 and confirm that there are no other analyte detection devices around as far as possible. After the user starts the remote equipment 103, the remote equipment 103 searches and identifies the nearby signal. If the remote equipment 103 recognizes only one first signal, it can be judged that the signal is the signal transmitted by the analyte detection device 102 to establish a communication connection, and a communication connection with the analyte detection device 102 is established through the link of the signal, without the user manually inputting or scanning the equipment code of the analyte detection device 102, it simplifies the process of establishing communication connection, avoids users from inputting or scanning wrong device code, and improves the user experience.

In other embodiments of the invention, before the analyte detection device 102 establishes a communication connection with the remote equipment 103, the remote equipment 103 prompts the user to confirm whether to connect, so as to improve the reliability of the communication connection between the analyte detection device 102 and the remote equipment 103.

In the embodiment of the invention, if the user is in a complex environment, that is, the remote equipment 103 recognizes multiple first signals, or multiple first signals and second signals, and the remote equipment 103 cannot judge the first signal transmitted by the analyte detection device 102 to establish a communication connection, in this case, the remote equipment 103 prompts the user to manually input or scan the device code of the analyte detection device 102 to establish a communication connection, In order to establish a communication connection with the analyte detection device 102.

In other embodiments of the invention, when the user is in a complex environment, the remote equipment 103 cannot judge the first signal transmitted by the analyte detection device 102 to establish a communication connection, and the remote equipment 103 prompts the user to change the operation location. The user needs to carry the analyte detection device 102 and the remote equipment 103 to other locations until the remote equipment 103 recognizes only one first signal, It can be judged that the signal is the signal transmitted by the analyte detection device 102 to establish the communication connection, and establish the communication connection with the analyte detection device 102 through the link of the signal, without the user manually inputting or scanning the equipment code of the analyte detection device 102, which simplifies the process of establishing the communication connection, avoids the user inputting or scanning the wrong equipment code, and improves the user experience.

In other embodiments of the invention, if the remote equipment 103 does not recognize the first signal within the effective range, it is judged that the analyte detection device 102 is not working normally. At this time, the remote equipment 103 sends an alarm or fault prompt to the user to prompt the user to check or replace the analyte detection device.

In the embodiment of the invention, the prompt of the remote equipment 103 may be one or more of the forms of audio, video or vibration. In one embodiment of the invention, when the prompt of the remote equipment 103 is audio, according to different prompt needs, the remote equipment 103 sends out “tick” prompt tones with different lengths and/or time intervals. In another embodiment of the invention, when the prompt of the remote equipment 103 is video, different text prompts are displayed on the display screen according to different prompt needs. In another embodiment of the invention, when the prompt of the remote equipment 103 is vibration, the remote equipment 103 vibrates with different lengths and/or time intervals according to different prompt needs.

To sum up, the embodiment of the invention discloses a system and method for realizing the communication connection of the analyte detection device. When the communication connection is not established or has been established, the analyte detection device transmits two different signals respectively, namely the first signal and the second signal. The remote equipment searches and identifies the nearby signals, and executes different communication connection procedures according to the type and quantity of the identified signals. When only one first signal is recognized by the remote equipment, it can be judged that the analyte detection device transmitting the signal is the analyte detection device to be established with communication connection, and the communication connection can be established directly without the user manually inputting or scanning the equipment code, which simplifies the process of establishing the communication connection, avoids the user inputting or scanning the wrong equipment code, and improves the user experience.

Although some specific embodiments of the invention have been detailed through examples, technicians in the field should understand that the above examples are for illustrative purposes only and are not intended to limit the scope of the invention. Persons skilled in the field should understand that the above embodiments may be modified without departing from the scope and spirit of the present invention. The scope of the invention is limited by the attached claims.

To the claims:

1. An analyte detection device communication system, comprising:
an analyte detection device, which is installed on a skin of an user, obtains an analyte parameter information in the user, and transmits signals to a remote equipment, wherein the signals at least comprise at least one first signal transmitted by the analyte detection device which hasn't established a communication connection with the remote equipment and a second signal transmitted by the analyte detection device which has established the communication connection with the remote equipment; and
the remote equipment, which is used to search and recognize nearby signals and execute communication connection procedures.

2. According to the analyte detection device communication system of claim 1, wherein the remote equipment is configured to execute a first communication connection program when the remote equipment recognizes the first signal, a number of the at least one first signal is only one, the remote equipment establishes the communication connection with the analyte detection device transmitting the first signal.

3. According to the analyte detection device communication system of claim 2, wherein before executing the first communication connection program, the remote equipment sends a prompt that needs to be confirmed by the user.

4. According to the analyte detection device communication system of claim 1, wherein the remote equipment is configured to execute a second communication connection program when the remote equipment recognizes the at least one first signal, a number of the at least one first signal is at least two, prompt the user to input an equipment code of the analyte detection device to be connected.

5. According to the analyte detection device communication system of claim 1, wherein the remote equipment is configured to execute a third communication connection program when the remote equipment recognizes the at least one first signal, a number of the at least one first signal is at least two, prompt the user to change an operation place until the remote equipment only recognizes the first signal whose number is one, and then execute a first communication connection program.

6. According to the analyte detection device communication system of claim 5, wherein an effective range of the first signal is 0˜10 m, and an effective range of the second signal is 0˜10 m.

7. According to the analyte detection device communication system of claim 6, wherein the effective range of the first signal is 0˜1 m, and the effective range of the second signal is 0˜10 m.

8. According to the analyte detection device communication system of claim 1, wherein the remote equipment is configured to send an alarm or fault prompt to the user when the remote equipment does not recognize the first signal.

9. According to the analyte detection device communication system of claim 3, wherein the prompt is in a form of one of audio, video and vibration.

10. According to the analyte detection device communication system of claim 1, wherein the first signal differs from the second signal in signal frequency, signal type, signal strength or/and signal format.

11. A method for realizing communication connection of analyte detection device, comprising:
providing an analyte detection device, wherein the analyte detection device which hasn't established a communication connection with a remote equipment transmits a first signal, and the analyte detection device which has established the communication connection with the remote equipment transmits a second signal;
providing the remote equipment, which is used for communication connection with the analyte detection device;
activating the analyte detection device to establish the communication connection;
transmitting the first signal;
starting the remote equipment 1;
searching and recognize nearby signals 1; and
executing a communication connection program.

12. According to the method for realizing communication connection of analyte detection device of claim 11, wherein the remote equipment is configured to execute a first communication connection program when the remote equipment recognizes the first signal, a number of the at least one first signal is only one, the remote equipment establishes the communication connection with the analyte detection device transmitting the first signal.

13. According to the method for realizing communication connection of analyte detection device of claim 12, wherein before executing the first communication connection program, the remote equipment sends a prompt that needs to be confirmed by the user.

14. According to the method for realizing communication connection of analyte detection device of claim 11, wherein the remote equipment is configured to execute a second communication connection program when the remote equipment recognizes the at least one first signal, a number of the at least one first signal is at least two, prompt the user to input an equipment code of the analyte detection device to be connected.

15. According to the method for realizing communication connection of analyte detection device of claim 11, in that, wherein the remote equipment is configured to execute a third communication connection program when the remote equipment recognizes the at least one first signal, a number of the at least one first signal is at least two, prompt the user to change an operation place until the remote equipment only recognizes the first signal whose number is one, and then execute a first communication connection program.

16. According to the method for realizing communication connection of analyte detection device of claim 11, wherein the remote equipment is configured to send an alarm or fault prompt to the user when the remote equipment does not recognize the first signal.

17. According to the method for realizing communication connection of analyte detection device of claim 11, wherein the analyte detection device without the communication connection comprises a newly delivered analyte detection device or a analyte detection device with a disconnected communication connection.

18. According to the method for realizing communication connection of analyte detection device of claim 11, wherein the first signal differs from the second signal in signal frequency, signal type, signal strength or/and signal format.
